(19) United States
(12) Patent Application Publication
(10) Pub. No.: US 2003/0054331 A1
(43) Pub. Date: Mar. 20, 2003

(54) Preservation of non embryonic cells from non hematopoietic tissues

(75) Inventors: John K. Fraser, Los Angeles, CA (US); Marc H. Hedrick, Encino, CA (US)

Correspondence Address: Michael J. Ram, KOPPEL, JACOBS, PATRICK & HEYBL, Suite 107, 555 St. Charles Drive, Thousand Oaks, CA, 91360, US

(73) Assignee: STEMSOURCE, INC.

(21) Appl. No.: 10/242,094

(22) Filed: Sep. 12, 2002

Related U.S. Application Data
(60) Provisional application No. 60/322,070, filed on Sep. 14, 2001.

(51) Int. Cl. A01N 1/02; C12N 5/08

(52) U.S. Cl. 435/2; 435/366

(57) ABSTRACT

The present invention is directed to stem cell and progenitor cell-containing populations derived from adipose tissue that are cryopreserved, the process for recovering and storing those cell populations, and the therapeutic uses of such populations upon thawing. It is further directed to the connective tissue matrix material, which may also be extracted from adipose tissue, preserved for subsequent therapeutic, structural, or cosmetic use in a mammalian patient.

This application claims benefit of U.S. Provisional Patent Application No. 60/322,070 filed Sep. 14, 2001.

The present invention is directed to a population of adipose tissue-derived cells, specifically adipose tissue-derived stem and progenitor cells, which are cryopreserved for subsequent use and to the therapeutic uses of said cryopreserved cells. It is also directed to the preparation and storage of connective tissue matrix material extracted from adipose tissue and to the therapeutic and cosmetic use of such material after storage. These cell populations and the matrix material can be used for therapeutic, structural, and/or cosmetic procedures on patients with various diseases and disorders. In a preferred embodiment cells that have been cryopreserved and thawed can be used for autologous (self) repair, reconstitution, reconstruction, and diagnostic applications. The invention also relates to methods for collection, processing, and cryopreservation of the stem and progenitor cells and the connective tissue of the invention. While primarily intended for human use the process described, and the materials recovered and preserved, also can be used in veterinary medicine, being applicable to other mammals.

### BACKGROUND OF THE INVENTION

Tissue Engineering

Tissue engineering is the term commonly applied to the technology of using the building blocks of biological tissues to regenerate, repair, or reconstruct damaged or deficient body structures or organs. For example, it is contemplated that a heart attack can be treated by growing new heart tissue or a slow healing bone fracture can be treated by application of bone-creating cells.

The core building blocks of tissue engineering (1) cells, preferably cells with high proliferative and differentiative capacity capable of making a large amount of many different tissues in a user-defined manner, (2) scaffolds providing a three dimensional structure upon which the tissue develops, such scaffolds being of natural, artificial, or composite materials, and (3) growth factors and chemical drugs which drive the cells to use the scaffold and generate the new or repaired tissue.

An objective of the invention is the extraction of adipose tissue, separation of desired components in that tissue and the subsequent storage of materials within the scope of the first two building blocks above, namely, adipose tissue-derived cell populations containing stem and progenitor cells and the connective tissue matrix material which is a natural scaffold.

### BACKGROUND

Stem Cells and Progenitor Cells

Stem cells are the master cells of the body (Blau H M, T R Brazelton and J M Weimann “The Evolving Concept of a Stem Cell: Entity Or Function?” Cell, 105, p829-841, (2001)). Stem cells from embryos or embryonic stem cells (ESCs) are known to become many if not all of the cell and tissue types of the body. These ESCs, also referred to as early fetal cells, not only contain all the genetic information of the individual but also contain the nascent capacity to become any of the 200+ different cells and tissues of the body. Ongoing research suggests that these cells have tremendous scientific and clinical potential (Weissman I L “Translating Stem And Progenitor Cell Biology To The Clinic: Barriers And Opportunities” Science 287, p1442-1446, (2000)).

However, ESCs have theoretic limitations to their use. If used clinically they would necessarily be derived from the embryonic form of one individual. When stem cells or tissues derived from that individual transplanted into another person, toxic immune suppressing drugs may be needed by the cell recipient to prevent rejection. In addition, cells from an individual can carry viruses or other rare but significant diseases to the recipient. Also, ESC-like cells (e.g. teratomas) are known to form tumors. Any tissues developed for use in patients from ESCs must not develop tumors and must have the innate ability to respond to the normal signals that tell tissues to grow, stop growing or heal.

Recently, non-embryonic or ‘adult’ stem cells have been identified and represent an important potential alternative to the clinical use of ESCs (Pittenger M F, Mackay A M, Beck S C, Jaiswal R K, Douglas R. Mosca J D, Moorman M A, Simonetti D W, Craig S, and Marshak D R “Multilieage Potential Of Adult Human Mesenchymal Stem Cells, Science, 284, p143-147, (1999); Zuk P A, Zhu M, Mizuno H, Huang J, Futrell J W, Katz A J, Benhaim P, Lorenz H P and Hedrick M H “Multilineage Cells From Human Adipose Tissue: Implications For Cell-Based Therapies”, Tissue Eng 7, p211-218, (2001)). These cells reside quietly in many if not all tissues, presumably waiting to respond to trauma or other destructive disease processes so that they can heal the injured tissue. Emerging scientific evidence indicates that each individual carries a pool of stem cells that may share with ESC's the ability to become many if not all types of cells and tissues.

Progenitor cells are an intermediate population between the stem cell and the mature cell. They are usually generally more restricted in their ability to generate multiple mature cell types and they are, by definition, considerably more restricted in their proliferative capacity with limited or absent ability to self-renew. Examples of progenitor cells include the CFU-GM and BFU-E cells of the hematopoietic system, and preadipocytes, and pre-osteoblasts of the mesenchymal system.

Adipose Tissue as a Source of Cells for Tissue Engineering

Adipose tissue has recently been found to be a source of stem cells, progenitor cells and matrix material suitable for therapeutic applications (Zuk et al, ibid; Huang, J I, S R Beanes, Zhu M, H P Lorenz, Hedrick M H and Benhaim P “Rat extramedullary adipose tissue as a source of osteochondrogenic progenitor cells” Plast Reconstr Surg. 109: p1033-1041, discussion, p1042-1043, (2002); Mizuno H, Zuk P A, Zhu M, Lorenz H P, Benhaim P and Hedrick M H “Myogenic Differentiation by Human Processed Lipoaspirate Cells. Plast Reconstr Surg. 109: p199-209; discussion 210-1 (2000)). Adipose tissue is also a rich source of vascular endothelial cells (Kern P A, Knedler A and Eckel R H “Isolation And Culture Of Microvascular Endothelium From Human Adipose Tissue” J Clin Invest 71, p1822-1829, (1983)) which may also play a role in tissue regeneration and engineering by promoting growth of new blood vessels and stimulating stem and progenitor cell growth (Hutley L J, Herington A C, Shurety W, Cheung C, Vesey D A, Cameron D P and Prins J B “Human Adipose Tissue Endothelial Cells Promote Preadipocyte Proliferation” Am J Physiol Endocrinol Metab, 281, p1037-1044, (2001)). Other tissue types contain such cells, but no other tissue can be extracted from a person in substantial quantities with so little morbidity. Suction-assisted lipoplasty (liposuction) is a very common procedure performed for aesthetic body recontouring. The American Society for Aesthetic Plastic Surgery reports that over 385,000 liposuction procedures were performed in the United States during 2001. At present the standard practice is to discard and destroy the tissue obtained following liposuction and other procedures for the removal of adipose tissue.

Adipose tissue-derived stem and progenitor cell populations have been shown to have the capacity to differentiate into numerous cell types (Zuk et al, ibid; Huang et al, ibid; Mizuno et al, ibid). Most importantly, although differentiation into cell lineages of mesodermal origin is the best characterized of this differentiation capacity, there is evidence for differentiation into endodermal and ectodermal lineages. As all cells of the human body derive from mesoderm, endoderm, or ectoderm these data indicate that adipose tissue-derived stem and progenitor cells have the potential to develop into any cell of the human body and thus have enormous therapeutic potential (WO00/53795, Published 14^{th} September, 2000). For example, bone, cartilage, muscle and fat are derived from mesoderm, nerve and skin cells are derived from ectoderm and various organs such as pancreas and bowel can be treated with endoderm cells. In addition to this potentially totipotent differentiation capacity, said cells have considerable proliferative capacity, being capable of undergoing in vitro culture through many, many passages (cycles). These twin abilities (differentiation and proliferation) make adipose tissue-derived stem and progenitor cells of value for tissue engineering and gene medicine.

However, the utility of stem cells goes beyond tissue engineering. Thus the proliferative and differentiative capacity of stem cells can be harnessed in gene transfer and gene therapy approaches in which the progeny of stem cells are used as gene/gene product delivery vehicles.

Cell Cryopreservation

As individual cells, a cell pellet or a suspension of cells is frozen and the fluid inside and outside the cell freezes there are numerous opportunities for cell damage due to fluctuations in local solute concentrations and formation of ice crystals within and around the cell as discussed in (Karlsson J O and Toner M, “Long-Term Storage Of Tissues By Cryopreservation: Critical Issues” Biomaterials 17, p243-256, (1996)). It has been found that the negative effects of these events can be minimized by use of cryoprotective additives and by controlling the rate of cooling. Certain cryoprotective additives act by permeating the cell wall reducing potentially harmful concentration differentials. Similarly, when cells are cooled slowly the rate of water efflux is sufficiently high to prevent excessive supercooling, promoting cell dehydration over intracellular ice formation. Different cooling rates have been used in different settings and some protocols use mathematical modeling to support use of a multistep cooling process. Also, many different cryoprotective agents have been described in the literature, incorporated herein by references (Pegg D E “The Current Status Of Tissue Cryopreservation”, Cryo Letters 22, p105, (2001); Karlsson J O and Toner M., ibid; Rowley S D “Hematopoietic Stem Cell Cryopreservation: A Review of Current Techniques” J Hematother. 1, p233, (1992)).

While no published history of cryopreservation of non-hematopoietic human stem and progenitor cells has been found, the proven clinical efficacy of hematopoietic stem cells (the cells responsible for the clinical efficacy of a conventional bone marrow transplant) in treatment for leukemia and certain inherited disorders has led to many studies directed at optimization of the cryopreservation of hematoporetic cells. Two studies have examined recovery of such cells from umbilical cord blood thawed after periods of up to 15 years of cryopreservation (Mugishima H, Harada K, Chin M, Suzuki T, Takagi K, Hayakawa S, Sato K, Klein J P and Gale R P “Effects Of Long-Term Cryopreservation On Hematopoietic Progenitor Cells In Umbilical Cord Blood”, Bone Marrow Transplant 23, P395-396, (1999); Kobylka P, Ivanyi P, and Breur-Vriesendorp B S “Preservation of Immunological and Colony-forming Capacities of Long-Term (15 years) cryopreserved Cord Blood Cells” Transplantation 65, p1275, (1998)). Another study by Re, et al showed clinical efficacy of cryopreserved bone marrow stem cells that had been stored for more than seven years (Re A, Vijayaraghavan K, Basade M M, He S, and Gulati S C “Long-Term Cryopreservation: Successful Trilineage Engraftment After Autologous Bone Marrow Transplantation With Bone Marrow Cryopreserved For Seven Years”, J Hematother 7, p185, (1998)).

Accordingly, cryopreservation of bone marrow and blood-derived hematopoietic stem cells is well established. Recently, the large scale banking of umbilical cord blood stem cells has been instituted and there are now several private cord blood banks and several dozen public, unrelated donor cord blood banks around the world. U.S. Pat. Nos. 5,004,681 and 5,192,553 describes umbilical cord blood stem cell banking.

Stem and Progenitor Cell Cryopreservation

As with all cell populations of the human body, adipose tissue-derived stem and progenitor cells age. (D'Ippolito G, Schiller P C, Ricordi C, Roos B A, and Howard G A “Age-Related Osteogenic Potential Of Mesenchymal Stromal Stem Cells From Human Vertebral Bone Marrow”, J Bone Miner Res 14, p1115-1122, (1999); Muschler G F, Nitto H, Boehm C A, and Easley K A “Age- and Gender-Related Changes In The Cellularity Of Human Bone Marrow And The Prevalence Of Osteoblastic Progenitors”, J Orthop Res 19, p117-125, (2001); O'Driscoll S W, Saris D. B., Ito Y, and Fitzimmons J S “The Chondrogenic Potential Of Periosteum Decreases With Age” J Orthop Res 19, p95-103, (2001); Long M W, Ashcraft E K Normalle D, and Mann K G “Age-Related Phenotypic Alterations In Populations Of Purified Human Bone Precursor Cells” J Gerontol A Biol Sci Med Sci 54, pB54, (1999); Mueller S M and Glowacki J “Age-Related Decline In The Osteogenic Potential Of Human Bone Marrow Cells Cultured In Three-Dimensional Collagen Sponges” J Cell Biochem 82, p583-590, (2001)). Cell aging is associated with decreased cell function. The process of cryopreserving these cells effectively places them in suspended animation. Specifically, at the temperatures typically used in cryostorage (−196° C.) there is insufficient energy for chemical reactions and thus no energy for metabolism, cell division, and the aging process. In theory, cosmic radiation represents the only source of damage or insult to cryopreserved cells (Karlsson, et al, ibid). Thus, an individual might reasonably choose to cryopreserve their stem cells at an early age to ensure a supply of younger, presumably more youthful cells for potential use in later life when the stem cells remaining in the body are older and of lower function. Beyond aging there are other insults that justify storage of stem cells outside the body. For example, there is ample evidence that conventional cancer chemotherapy and radiotherapy have a negative effect on the function of stem cells and that the deleterious effects of these treatments on stem cells might be associated with some of the side effects of therapy (Kashyap, et al, “Effects Of Allogencic Bone Marrow Transplantation On Recipient Bone Mineral Density: A Prospective Study”, Biol Blood Marrow Transplant 6, p344, (2000); Banfi A, Podesta M, Fazzuoli L, Sertoli M. R, Venturini M, Santini G, Cancedda R, and Quarto R “High-Dose Chemotherapy Shows A Dose-Dependent Toxicity To Bone Marrow Osteoprogenitors: A mechanism for Post-bone Marrow Transplantation Osteopenia”, Cancer 92, P2419, (2001); Galotto M, Berisso G, Delfino L, Podesta M, Ottaggio L, Dallorso S, Dufour C, Ferrara G. B., Abbondandolo A, Dini G, Bacigalupo A, Cancedda R, and Quarto R “Stromal Damage As Consequence Of High-Dose Chemo/Radiotherapy In Bone Marrow Transplant Recipients”, Exp Hematol 207, P1460, (1999)). Hence, a patient about to undergo such procedures might reasonable choose to have stem and progenitor cells extracted and stored so the cells are not subjected the effects of chemotherapy or radiotherapy.

Adipose Tissue-Derived Connective Tissue Matrix Material

Adipose tissue is a loose aggregation of mature adipocytes and other cell populations supplied by a rich blood supply and held together by a complex connective tissue matrix. This matrix is composed largely of collagen, a key structural protein. It has value in reconstruction of tissue and as a scaffold for cells to grow on in reconstituted organs or repairing tissue damage.

### SUMMARY OF THE INVENTION

A population of adipose tissue-derived cells that includes stem and progenitor cells is isolated and cryopreserved, for therapeutic uses of such cells upon thawing. In addition, adipose tissue-derived connective tissue matrix material can be extracted and stored for later therapeutic use.

In particular, the present invention relates to the isolation, preservation and therapeutic use of cryopreserved adipose-derived stem cell, progenitor cell-containing populations and matrix materials in therapeutic, structural, or cosmetic applications for repair, reconstitution, and/or reconstruction. Stem and progenitor cells which contain a heterologous gene sequence for use in gene therapy in delivering replacement or novel gene sequences is also contemplated.

In a preferred embodiment of the invention, cells that have been cryopreserved and thawed can be used for autologous (self) reconstitution. In a further preferred embodiment, the connective tissue matrix material may be used for autologous (self) or allogeneic (non-self) repair and reconstruction.

The invention also relates to methods of collection, processing, and cryopreservation of the connective tissue matrix and the stem and progenitor cell-containing population of the invention.

The invention further relates to unique compositions of matter, namely, cell population and matrix material isolated by the processes and methods described herein.

### DESCRIPTION OF THE FIGURES

FIG. 1 is a schematic diagram of a collection device with partial cutaway canister for use in collecting adipose tissue.

FIG. 2 is a partially cutaway view of the cannister of FIG. 1.

FIG. 3 is a micrograph (original magnification=100×) of chondrocytes (cartilage cells) formed in culture from adipose tissue-derived cell population after cryopreservation and thawing according to the methods herein. (Staining is with Alcian Blue showing as dark gray in this image, a standard reagent used to stain for cartilage-type cells).

FIG. 4 is a micrograph (original magnification=400×) of rat adipose tissue-derived cells processed and stored according to this invention and then thawed and implanted into a rat heart (animal sacrificed five days after implantation). Clusters of implanted cells are marked by arrows)

FIG. 5 is a micrograph (original magnification=400×) of human adipose tissue-derived cells processed and stored according to this invention and then thawed and implanted into the bladder of an immunodeficient mouse. The animal was sacrificed 30 days after implantation. Cells were labeled with Bromodeoxyuridine prior to implantatation. Labeled cells appear with dark gray/black nuclei throughout this image and examples are highlighted with arrows.

FIG. 6 is a micrograph (original magnification=400×) of human adipose tissue-derived cells growing on a scaffold of adipose tissue-derived matrix prepared and stored according to this invention. Mature, fat-laden adipocytes are highlighted with arrows.

### DETAILED DESCRIPTION

The present invention is directed to cryopreserved stem and progenitor cell-containing populations derived from adipose tissue, the therapeutic uses of such cells upon thawing and the methods and processes for obtaining such materials. It is further directed to connective tissue matrix material derived from adipose tissue and the extraction, preservation and storage of these materials.

In particular, the present invention relates to the collection, processing, and storage of adipose tissue-derived stem and progenitor cell-containing populations, and connective tissue matrix for later use. These processes are collectively referred to as the banking of adipose tissue-derived cells and material. A preferred embodiment is directed to post-storage use of these products in the individual from whom the cells and material were originally derived, said individual diagnosed as having a disease state treatable by delivery, systemically or directly to particular sites in the body, of stem and/or progenitor cells. This embodiment may be described as autologous banking and implantation of adipose tissue-derived stem and progenitor cell-containing populations, and/or connective tissue matrix.

It has now been demonstrated that the number and capacity of stem cells declines with age Therefore, it is not unreasonable for individuals to bank their own stem cells and associated products while they are relatively young so that they have a source of stored, viable, young stem cells available to them if and when they need them as they grow older. Various different disease states where delivery of stem cells or progenitor cells is likely to be medically beneficial include, but are not limited to osteoporosis, myocardial infarction, bone fracture, cartilage damage through accident or arthritis, neurological damage or disease, and aesthetic or trauma related plastic surgical applications. This list is not intended to limit the utility of the invention as there are numerous medical conditions where stem cell and/or progenitor cell delivery is beneficial, and new applications thereof are being discovered daily. The advantage of banked cells include the fact that as autologous (self) cells they carry no risk of introduction of a transfusion or transplant-related disease and there is no risk of rejection or graft versus host disease, which are significant risks for allogeneic cells (cells donated by another person). In addition, in studies of hematopoietic stem cells (stem cells which make blood cells and which are derived from bone marrow, peripheral blood, or umbilical cord blood) application of autologous cells is considerably more efficient than that of cells derived from another person and many-fold fewer cells are required for hematopoietic reconstitution (development of functional blood cell production).

The method of cell recovery, isolation and preservation may include, but is not limited to some or all of the following steps or stages: (a) collection of adipose tissue; (b) packaging of the tissue at the collection site; (c) transport of the tissue to a processing facility (Bank); (d) processing adipose tissue to extract stem and progenitor cell-containing populations and to isolate the cellular connective tissue matrix material; (e) inspection and testing of cells and matrix material; (f) storage; (g) recovery of cells and matrix material from the stored state; (h) examination of cells and matrix material recovered for clinical therapy; (i) optional ex vivo manipulation of the cells; (j) a quality system which functions to maximize the efficiency and quality of all Bank activities; and (k) therapeutic uses in repair, reconstitution, and reconstruction of human cellular and cellular systems and tissues. One skilled in the art will recognize that the order of performing certain of these steps may be varied.

The practice of this invention falls under the general heading of Tissue Banking and, thus, the practice may be required to conform with the requirements of professional bodies addressing such issues, such as the American Association of Tissue Banks and the American Association of Blood Banks. Governmental agencies such as the US Food and Drug Administration may also mandate standards applicable to the process and uses set forth herein. However, the novelty of this invention is such that no standards currently exist that are directly applicable.

Collection of Adipose Tissue

Adipose tissue is the source of the stem and progenitor cell-containing populations, and connective tissue matrix material (the preferred desired components) contemplated by the present invention. However, other components of the adipose tissue present in the remaining portion (the discardable components) may also have medical value and may be used for other applications. Adipose tissue can be obtained by any method known in the art. For example, adipose tissue may be collected during various routine liposuction procedures such as performed for the purpose of aesthetic body recontouring. The tissue can be collected into a device designed for and dedicated to the purpose of collecting the tissue for banking or it can be collected into the usual devices used for collection of lipoaspirate by personnel performing liposuction procedures.

The method described herein for collecting stem cells is superior to other methods using sources such as bone marrow, skin, or skeletal muscle. It can be performed in a closed system and, as specified above, lipoaspiration obtains a much larger amount of tissue without the side effects and risks associated with bone marrow harvest or skin and muscle biopsy.

Adipose tissue may also be collected in conjunction with surgical excision of skin and associated adipose tissue. Examples include tummy tuck, excisional lipectomy or incidental to other surgical procedures.

Collection of adipose tissue is preferably made under sterile conditions. Immediately upon collection, the adipose tissue and/or collection device should be sealed and transported to a processing facility. It is anticipated that, in some settings, the collection facility and the processing facility may be in close proximity permitting a simplified sealing and transportation of the product from one area to another.

Amount of Adipose Tissue Collected

Aspirated adipose tissue contains varying amounts of stem cells, progenitor cells, matrix material, blood, serum, lipids, adipocytes, vascular endothelial cells, vascular smooth muscle cells, and pericytes (Hausman G. J. and R. L. Richardson “Cellular And Vascular Development In Immature Rat Adipose Tissue” J Lipid Res 24, p522-532 (1983); Greenwood M. H., “Adipose Tissue: Cellular Morphology And Development” Ann Intern Med 103, p996-999, (1985); Kern, et al, ibid).

In a first embodiment, at least about 400 g of adipose tissue is obtained. Practical experience indicates that 400 g of adipose tissue yields approximately 100 million nucleated cells. However, smaller and larger amounts of adipose tissue may also be acceptable, and indicated under some circumstances. For example, it may be acceptable or preferable to harvest larger amounts of tissue from obese or overweight individuals than from lean persons.

As set forth in Table 1, while more tissue can be obtained from obese persons than from those of normal body mass index, the total cell yield in the adipocytes (fat cell)-depleted product obtained from a routine lipoplasty procedure is not significantly different. This is because lean persons have more non-adipocytes per gram of adipose tissue than obese persons.

One consequence of these observations is that the utility of this invention is not limited by the body mass index of the individual from whom the adipose tissue is extracted.

Furthermore, adipose tissue-derived stem cells and progenitor cells can be multiplied in culture, before or after cryopreservation, thus considerably expanding the number of cells available for therapy.

TABLE 1
Effect of Body Mass Index on Tissue and Cell Yield

| | Amount of Tissue | |
|---|---|---|
| Body Mass Index Status | Obtained (g) | Total Cell Yield (x107) |
| Normal | 641 ± 142 | 2.1 ± 0.4 |
| Obese | 1,225 ± 173 | 2.4 ± 0.5 |
| p value | 0.03 | 0.6 |

In an exemplary collection procedure, a collection device, packaged in a sterile container, can be used. In one particular embodiment, the collection kit 10 can consist of: a sterile collection cannister 12 composed of medical grade materials and containing a medical grade filter 14 material allowing free lipid, solutions used in lipoplasty, and blood to flow through while retaining adipose tissue fragments in a cell collection portion 15 of the collection device. The cannister 12 has a port 16 for connection of tubing 18 attached to a suction source on one side of the filter 14 material and a second port 20 connected by flexible tubing 22 to a sterile, single use, disposable liposuction cannula 24. The cannister 12 may also contain other ports 26, 28 and connection mechanisms 30, 32 for permitting ingress and egress of materials such as those used during tissue processing. The device would also have attached a label similar to labels affixed to blood collection bags to label the device and permit addition of written and adhesive label-based information about the tissue collection (for example, donor identification, date, time). The cannister may also have a means or indicia (not shown) for measuring the amount of tissue retained by the filter. Clamps or valves 34 can be attached to tubing 18, 22 or included in any of the various tubing or access ports 16, 20, 26, 28, 30, 32 to control flow of fluids or applied vacuum level.

A typical filter material suitable for retaining at least about ninety percent (90%) of the stem cells and progenitor cells is a polyester mesh of 200 μm thickness with a pore size of 265 μm and 47% open area.

Sterilization of the cannister and attached components prior to use can occur by any technique known in the art, including but not limited to beta- or gamma-irradiation, autoclaving of suitable materials in a steam sterilizer, ETO exposure, etc. For example, in a preferred embodiment sterilization is performed by gamma-irradiation at a dose validated to provide sterility in accordance with predetermined requirements such as those mandated by FDA and other regulatory bodies.

The collection kit 10 may be placed in the surgical field in advance of a lipoaspiration, to afford ready availability.

It is anticipated that many alternative components, materials, and devices will occur to one skilled in the art and the embodiment above is provided purely as an example and is not intended to limit the scope of the invention and the claims included in this disclosure. Suction-assisted lipoplasty (liposuction) performed by qualified medical personnel for the purpose of aesthetic body recontouring, is the preferred source of adipose tissue. This procedure routinely yields in excess of 100 g of lipoaspirate although there is no lower or upper limit to the amount of tissue that may be processed and stored under this invention. Other lipoplasty techniques such as power-assisted lipoplasty and ultrasound-assisted lipoplasty may also be used to collect adipose tissue. Similarly, techniques using lipectomy (surgical excision of adipose tissue) alone or in the context of a larger surgery such as abdominoplasty are within the scope of this invention.

Following preparation of the patient for the liposuction procedure the collection device is attached to the liposuction pump and the cannula is inserted into the donor in accordance with the practice of that Physician.

Maintaining usual sterile precautions, the liposuction procedure is performed until either the procedure is complete or the collection device is full. The collection device 10 is then sealed to maintain the sterility of the interior compartment containing the tissue. The collection cannister, with or without connected tubing specific instructions for operating the collection device described above are can then be separated from the vacuum pump and shipped to a processing facility.

Instructions for Using the Collection System

Exemplary instructions for operating the collection device described above are:

1. Prepare the patient as you usually do for traditional lipoplasty.

2. Connect the liposuction pump (with conventional trap and in-line microbial filters) to the hose 18 leading from the Collection Container 12.

[symbol] Note: the Collection System is designed to collect tissue fragments and aggregates. Saline, blood, and free lipid will pass freely through the filter and Collection Container. Consequently, the Collection Container MUST be placed upstream of a conventional trap with in-line microbial filters to prevent contamination of the suction pump.

3. Ensure that the valve 34 on the tubing 18 attached to suction port 16 of the Collection Container 12 and the tubing 22 to cannula 24 are open.

4. Confirm the orientation of the cannula suction tip 24 and use the cannula as a regular liposuction cannula to remove adipose tissue.

5. After up to 1.2 liters of adipose tissue has been collected within the collection container, close the valves 34 on the inlet and outlet tubing to fully seal the device.

[symbol] The sterility of the system depends on effective sealing.

6. Remove (by cutting) the tubing on both sides of the device approximately 2 inches outside of the valves.

7. Apply a Client Identification Barcode Label to the Collection Container 12. Recording the date and time of the procedure on the Label.

8. Apply the remaining Client Identification Barcode labels to worksheets and complete these worksheets. Return one copy of this form with the lipoaspirate tissue, keep a copy for your records, and give the 3^{rd} copy to the patient.

In a preferred embodiment the Collection Container 12 is sealed and shipped to the processing site. Optionally the tissue may be washed by using suction to pass a sterile washing solution (for example isotonic saline) through the device and tissue. Preservative agents designed to enhance stem cell viability during shipment may also be added to the tissue.

Besides the patient information, the date and time the liposution was performed, it may also be appropriate to record the sites from which tissue was obtained and other medical data about the donor (age, gender, medical history, etc.).

When the tissue collection site and the tissue processing site are in close proximity, there may not be a need for packing the collection container for shipment.

However, when the adipose tissue must be transported a distance, it should be packaged in a manner that protects individuals engaged in transport from exposure to potentially biohazardous human material. It must also maximize retention of viability of the product during transport.

Protection of persons engaged in transport is controlled by adequately sealing the collection device or by transferring the adipose tissue into a more easily sealed container, said container being sterile. The primary container is then packaged in secondary and tertiary containers validated to retain seal under the worst conditions reasonably anticipated to occur during transit.

Protection of tissue viability is achieved by ensuring rapid, efficient transport to the processing facility. Temperature control may also be applied to maximize cell viability.

Accordingly, the collection cannister should be placed in an insulated container. Packing is supplemented with any temperature control known to the art (for example, wet ice, frozen gel packs) that will maintain a suitable temperature throughout the anticipated duration of transport. In a preferred embodiment, the refrigerant and insulated container should be validated to provide predetermined temperature control functions (for example, they should hold temperature below a predetermined point for at least a predetermined time).

Other embodiments of sealing and packaging will occur to one skilled in the art and may be employed as required to conform with regulatory requirements or quality system improvements.

In order to preserve the viability of the stem cell compartment of the adipose tissue between the time of collection and the time of processing it is essential that the conditions of transport be controlled. In addition, there are regulatory requirements regarding the shipment of untested human biologic material.

Processing of Adipose Tissue

Processing of adipose tissue comprises a series of washing and extraction steps in which adipose tissue fragments are optionally washed to remove residual free lipid and blood before the cell population of interest is extracted. Given the nature of adipose tissue a suitable approach is to disaggregate the tissue by disrupting the connective tissue. The low buoyant density of lipid is such that free lipid and lipid laden mature adipocytes will float in common isotonic buffered solutions while the adipose tissue-derived stem cells and progenitor cells will sediment allowing easy separation of the more abundant adipocyte population. Suitable buffered solutions include, but are not limited to, Plasma Lyte A® (Baxter Inc.), Normosol R (Abbott Laboratories), and Phosphate Buffered Saline).

Tissue disaggregation may be performed by any means known to the art including but not limited to digestion with proteolytic enzymes, such as collagenase, trypsin, or papain, mechanical disaggregation using mincing or shear force, or other means. A combination of approaches such as enzymes along with mechanical disruption and/or combinations of enzymes including non-proteolytic enzymes such as DNase and lipase may also be applied.

Disaggregation allows release of cells from the adipose connective tissue matrix. Mature adipocytes can then be removed by equilibrium density centrifugation, buoyant density flotation, or other means. The remaining adipocyte-depleted cell population may then be concentrated by various means including, but not limited to, centrifugation, spinning membrane separation, differential adherence and elution to solid phase structures such as antibody-coated beads, fluorescence activated cell sorting, or other separation means, or a combination thereof.

Further fractionation of the population of cells obtained from adipose tissue (and the adipocyte depleted cell population) may be performed. Care should be taken to ensure maximal retention of the cell type of interest. Many cell separation technologies are known to the art and could be employed to further manipulate the cell population which is separated from the adipose tissue.

For example, removal of residual red blood cells may be achieved by exploiting the osmotic sensitivity of red blood cells exposing the population to a hypotonic shock in which red blood cells are lysed. Centrifugation approaches such as equilibrium density centrifugation or centrifugal elutriation could be employed as well as antibody-based approaches such as immunomagnetic cell selection, fluorescence-activated cell sorting, or panning techniques. Positive selection, negative selection, and combinations of both are included within the scope of this invention.

Adherence to plastic as well as cell culture approaches may be used as part of cell fractionation and/or processing.

Fractionation and enrichment of the population to the extent of separating stem cells from progenitor cells can be accomplished by cell culture, antibody-mediated positive and/or negative selection, differential density centrifugation, centrifugal elutriation, or other means known to the art. However, any means or combination of means known to the art to create pure or enriched populations may be used.

Adipose tissue-derived connective tissue matrix material may be extracted either directly from whole adipose tissue or from tissue from which the stem and progenitor cell population has been extracted. In a preferred embodiment the matrix material is largely retained within the cell collector portion 15 (in the filter enclosed area) following release of the stem and progenitor cell-containing cell population from the collection portion 15 and removal of this cell population for concentration and cryopreservation. Extraction of matrix material may then be performed by various different means. Examples include, lysis of mature adipocytes by exposure to hypotonic shock (for example sterile distilled water), by extraction with ionic or non-ionic detergents such as sodium deodecyl sulfate, triton X-100, Tween 20, by extraction with organic solvents such as acetone or isopropyl alcohol. Combinations of these extractants may be employed. For example, cells may be lysed with water, residual cellular material extracted by washing with detergent, and then residual lipid extracted using acetone.

In a preferred embodiment the adipose tissue is washed with sterile, buffered isotonic saline and incubated with collagenase at a collagenase concentration, temperature and time sufficient to provide adequate disaggregation. Suitable solutions have Collagenase concentrations from about 10 μg/ml to about 50 μg/ml and are incubated at from about 30° C. to about 38° C. for from about 20 minutes to about 60 minutes. These parameters will vary according to the source of the Collagenase enzyme, optimized by empirical studies, in order to validate that the system is effective at extracting the desired cell populations. A particular preferred concentration, time and temperature is 20 μg/ml Collagenase (Blendzyme 1, Roche) incubated for 45 minutes, at about 37° C. In a particular preferred embodiment the Collagenase enzyme used is material approved for human use by the relevant authority (for example, the US Food and Drug Administration). The collagenase used should be free of micro-organisms and contaminants such as endotoxin.

Cell and/or matrix material processing can be performed manually or in an automated fashion incorporating some or all of the steps listed above.

In a preferred embodiment minimization of the risk of contamination of the cell population with an adventitious microorganisms or with cells from another donor being processed at the same time is facilitated by use of a closed processing system.

Reagents should be added to, and waste and cells removed from, the collection device using approaches which maintain a sterile, closed fluid pathway. This includes addition of buffered isotonic solutions for washing the tissue, addition of enzymes used to disaggregate the tissue, and removal of the extracted cell population after digestion.

In a preferred embodiment, this is achieved by use of sterile connecting devices to connect closed, sterile tubing leading from the collection device to another piece of closed, sterile tubing leading from a bag of buffered saline. Similarly, waste fluid created during washing is removed from the collection device through closed sterile tubing that is connected to closed, sterile tubing leading to a closed, sterile waste bag. Cells released from the adipose tissue during processing are likewise removed from the collection device through sterile closed tubing connected to closed, sterile tubing leading to second closed sterile container. In this embodiment, the waste container and the cell collection container are flexible, sterile plastic bags. The cell collection bag is then placed in a centrifuge and cells are sedimented at 400×g for 10 minutes creating a stable cell pellet.

The sterile, closed tubing leading from this bag can then be connected to sterile, closed tubing leading to a second waste bag allowing transfer of the cell-depleted solution and retention of the cell pellet. The cell pellet can then be resuspended and prepared for cryopreservation. Finally, the bag containing the cells can be connected to sterile, closed tubing leading to a suitable cryopreservation container.

In this way, the cells can be maintained entirely within a closed, sterile fluid pathway from the point of collection to the point of cryostorage.

Adipose tissue-derived matrix material can be processed in the same general approach of serial closed sterile tubing connections. The connective tissue matrix is extracted directly from the tissue without first extracting the stem and progenitor cell-containing population or by extracting the glutinous waste material present at the end of tissue digestion.

In a preferred embodiment, efforts are made to utilize reagents and systems that are approved for human use by the relevant authorities. This is in order to promote donor safety and improve compliance with relevant regulations. Where a needed reagent is not available in a form that is approved for human use validation studies should be performed to ensure that the reagent or component meets predetermined suitability criteria. Such criteria could include purity analysis, endotoxin content, sterility, etc.

Sterile isotonic buffered solutions that are approved for human use are very common in medical practice and are available as a commodity from several manufacturers and distributors. Similarly, sterile closed bags bearing sterile closed tubing suitable for connection using a sterile connection device are commonly available as part of blood banking. Flexible plastic bags suitable for cryopreservation are also in common use in bone marrow transplantation programs and are available for purchase.

Typical testing performed on the cells and matrix material recovered include, but are not limited to:

Cell Population at the End of Processing

Total yield of cells

Sterility of cells

Viability of cells

Content of subpopulations within the final product including but not limited to:

endothelial cells

stem cells

progenitor cells

blood cells

residual mature adipocytes

Matrix Material at the End of Processing

Collagen Content

Sterility

Collagen integrity

These tests and inspections are conducted using procedures known to the art. For example, cell viability can be determined by vital dye exclusion, stem cell content can be determined by growth of CFU-F in a validated clonogenic assay system or by use of cell surface phenotyping using immunodetection, and collagen integrity can be determined by transmission electron microscopy. These assays are listed by way of example and are not intended to be an exhaustive list of potential assays as alternatives are known to those skilled in the art.

Cell Cryopreservation and Storage

As indicated above, cell freezing is destructive to the cells in the absence of appropriate additives and a validated cryopreservation process. It has also been found that the stem and progenitor cells must be separated from the fatty tissue and other biological materials before freezing. Attempts to cryopreserve adipose tissue and separate the stem cells after thawing has not produced useful amounts of viable stem cells.

Cryoprotective additives fall into two general categories; permeating cryoprotectants, which can pass through cell membranes, and non-permeating cryoprotectants. Examples of permeating cryoprotectants include, but are not limited to, dimethyl sulfoxide (DMSO), glycerol, and 1,2-propanediol. Examples of non-permeating cryoprotectant include, but are not limited to, hydroxyethy starch, albumin, and polyvinyl pyrrolidone.

The most commonly employed permeative cryoprotectant is DMSO, which is often used in combination with a non-permeative agent such as autologous plasma, human serum albumin, and/or hydroxyethyl starch.

In a preferred embodiment, the particular cryoprotectant additive(s) used is validated to assure adequate recovery of cells after thawing.

Recovery of viable cells following cryopreservation can be optimized by controlling the rate of cooling during the freezing process. The most common cooling protocols maintain a constant cooling rate of −1° C. to −3° C. during the critical phase of cooling which is from initiation to about −50° C.

This rate can be achieved by various means including but not limited to use of computer-controlled rate freezing, immersion in baths of cooled organic solvents, or placement within a mechanical freezer. In a preferred embodiment, the ability of the system chosen will be validated to generate the predetermined cooling rate. However, the cooling rate chosen may exceed the parameters specified above (−1 to −3° C. from start to −50° C.) provided the rate employed is validated to provide adequate recovery of cells after cryopreservation.

Following cooling of the cells to a suitable temperature, the sample may be transferred to a long term storage vessel. In a preferred embodiment this vessel will take the form of a vacuum jacketed liquid nitrogen dewar in which the sample can be either immersed in the liquid phase or held in the vapor phase of the chamber. Such vessels are available through a number of manufacturers and distributors being commonly used in agricultural (storage of animal sperm and ova) and medical (human sperm banking and bone marrow transplant programs) applications.

In a preferred aspect, the processed cells are resuspended in a buffered, isotonic solution (such as Plasma-Lyte A, Baxter) supplemented with 5% (by volume) human serum albumin (American Red Cross Blood Services, Washington D.C.) and cooled to 4° C. Dimethyl sulfoxide (DMSO) is then added as a cryoprotective agent to obtain a final concentration of from about 5% to about 20% preferably 10% DMSO. Use of up to 20% by volume final DMSO concentration may be applied, provided that appropriate validation studies are performed to demonstrate that equivalent or superior post-thaw recovery of cells of interest is achieved. DMSO is then added slowly (over approximately 15 minutes), for example, using a syringe pump with constant mixing of the cells during addition using cold packs to retain the temperature of the cells at 4° C.

Following addition of DMSO, the cells are transferred in a sterile manner into a flexible plastic bag suitable for long term storage in liquid nitrogen. The bag is sealed and placed in a cassette, such as an aluminum enclosure. This cassette is placed in a freezer which cools the cells from 4° C. to −50° C. at a controlled rate of −1° C. per minute. The temperature is then reduced to −90° C. at a rate of about −10° C. per minute. The cassette is then placed in the vapor phase of a liquid nitrogen dewar.

In a preferred embodiment, the cells are cryopreserved in multiple aliquots (by using more than one cryostorage container or using a cryostorage container that is divided into multiple compartments). This allows recovery of a portion of the frozen cells without compromising the storage conditions of the remainder.

Examples of alternatives cryopreservantion techniques include; storage in the liquid phase of nitrogen, storage in a mechanical freezer, storage in one or more cryotubes rather than in cryobags, use of higher or lower final concentrations of DMSO, etc. Similarly, embodiments using systems that do not maintain material at all times within a sterile, closed fluid pathway, or indeed where the material is at no time in such a pathway may be employed.

Adipose tissue-derived matrix material is largely composed of collagen, a very stable protein that is insoluble in water at neutral pH. This material can be stored by freezing the material, either in suspension or as a dry pellet, and storing at sub-zero temperatures, by freeze drying to a residual water content of between 0.5%-5% water by mass and storing at ambient temperature (<25° C.), or by other means known to the art. The material may be stored as a fully processed material that has been made ready for use or as an intermediate product of manufacture.

Suitable storage containers include, but are not limited to, storage in pre-loaded syringes, glass ampoules, tubes or flexible plastic bags.

The stability of the matrix material is such that storage conditions are not as critical and that multiple means for long and short-term storage will be obvious to one skilled in the art.

Recovery of Cryopreserved Cells

The conditions under which the temperature is raised and the cells are thawed is also critical. For example, very small intracellular ice particles can induce nucleation and recrystallization. Similarly, the osmotic stresses observed during freezing can also be a source of cell damage during thawing. Further, there is evidence to suggest that the cryoprotectant DMSO is toxic to cells above 4° C. (Hak A. M., F. G. Offerijns and C. C. Verheul “Toxic Effects Of DMSO On Cultured Beating Heart Cells At Temperatures Above Zero” Cryobiology 10, p244-250, (1973)). Thus exposure to this agent after thawing should be minimized.

Additionally, the use of a sterile, closed system and approved reagents is a preferred aspect of application of this invention.

In a preferred embodiment the cassette containing the cells is removed from the liquid nitrogen storage vessel and the freezer bag is removed from the cassette and placed in a sterile, flexible outer container (for example a sterilized ziploc-style plastic bag). This bag is sealed and immersed in a 37° C. waterbath to raise the temperature to about 4° C. The bag is gently manipulated during thawing to ensure uniform transfer of heat throughout the material during warming until the consistency of the material inside the bag is slush-like. At that point, the cryobag can be removed from the waterbath and the outer container and placed on cold packs while tubing or ports leading from the bag are connected to sterile, closed tubing leading to a closed, sterile container. This container can contain a buffered, isotonic solution supplemented with a protein source such as 5% human serum albumin. Cells are then transferred through the tubing from the cryobag to closed, sterile container holding the albumin solution. This receiving container may then be placed in a centrifuge to pellet the cells. Excess albumin solution and the DMSO may then be removed from the cells by expression of the liquid phase into a second sterile, closed bag connected by means that preserve a sterile, closed fluid system. The cells may then be resuspended in additional isotonic solution for delivery to the patient.

If, as specified in a particular embodiment above, the cells are stored in multiple cryostorage containers or in multiple compartments of a cryostorage container, only those bags or compartments needed at the time of recovery need to thawed.

Alternative embodiments which do not use washing and centrifugation to eliminate DMSO, which use alternate washing conditions or solutions, such as supplementation with an alternative source of protein, or which do not use a closed, sterile fluid pathway are also possible within the scope of this invention.

Additional compositions may be added to the washing solution to enhance cell recovery. For example, cells that have been ruptured during cryopreservation will often release their DNA into the medium. The highly charged, polymeric nature of DNA is such that it can cause cells to clump together. Thus, agents such as DNase, (the enzyme which cleaves DNA) may be added to minimize cell clumping.

Horwitz et al have described the results of transplanting human bone marrow-derived osteoprogenitor cells in patients with Osteogenesis Imperfecta, an inherited disease of bone production (Horwitz E M, Prockop D J, Gordon P L, Koo W W, Gordon P L, Neel M D, Sussman M, Orchard P, Marx J C, Pyeritz R E and Brenner M K “Transplantability and Therapeutic Effects Of Bone Marrow-Derived Mesenchymal Cells In Children With Osteogenesis Imperfecta” Nat Med 5, p3009, (1999); Horwitz E M, Prockop D J, Gordon P L, Koo W W, Gordon P L, Neel M D, McCarville M E, Orchard P J, Pyeritz R E and Brenner M K “Clinical Responses To Bone Marrow Transplantation In Children With Severe Osteogenesis Imperfecta” Blood 97, p1227, (2001)). In these studies, patients received a transplanted nucleated cell dose of 3.2 billion cells. Data published by Muschler et al indicates that this 3.2 billion of nucleated marrow cells contains approximately 176,000 osteoprogenitor cells (CFU-AP) (Muschler G F, Nitto H, Boehm C A and Easley K A “Age-And Gender-Related Changes In The Cellularity Of Human Bone Marrow And The Prevalence Of Osteoblastic Progenitors” J Orthop Res 19, p117, (2001)).

In animal studies, Orlic et al (Orlic D, Kajstura J, Chimenti S, Bodine D M, Leri A and Anversa P “Mobilized Bone Marrow Cells Repair The Infracted Heart, Improving Function And Survival” Proc Natl Acad Sci USA 98, p10344, (2001); Orlic D, Kajstura J, Chimenti S, Bodine D M, Leri A and Anversa P “Transplanted Adult Bone Marrow Cells Repair Myocardial Infarcts In Mice” Ann NY Acad Sci 938, p221, (2001); (Orlic D, Kajstura J, Chimenti S, Jakoniuk I, Anderson S M, Li B, Pickel J, McKay R, Nadal-Ginard B, Bodine D M, Leri A and Anversa P “Bone Marrow Cells Regenerate Infracted Myocardium” Nature 410, p701, (2001)) have demonstrated the potential for stem cells to engraft damaged heart muscle following a myocardial infarct and that such engraftment is associated with improvement in heart function. In one study his group demonstrated that stem cells conferred an improvement in survival (Orlic, et al, “Mobilized Bone Marrow Cells Repair The Infracted Heart, Improving Function And Survival” Proc Natl Acad Sci USA 98, p10344, (2001)). Orlic et al's results from animal studies using partially-purified stem cell fractions were realized with injection of 24,000 cells which represented approximately 1% of the starting whole bone marrow. Published data obtained using cryopreserved blood and marrow-derived stem cells have indicated that blood and bone marrow derived stem cells can retain stem cell viability.

### EXAMPLE 1

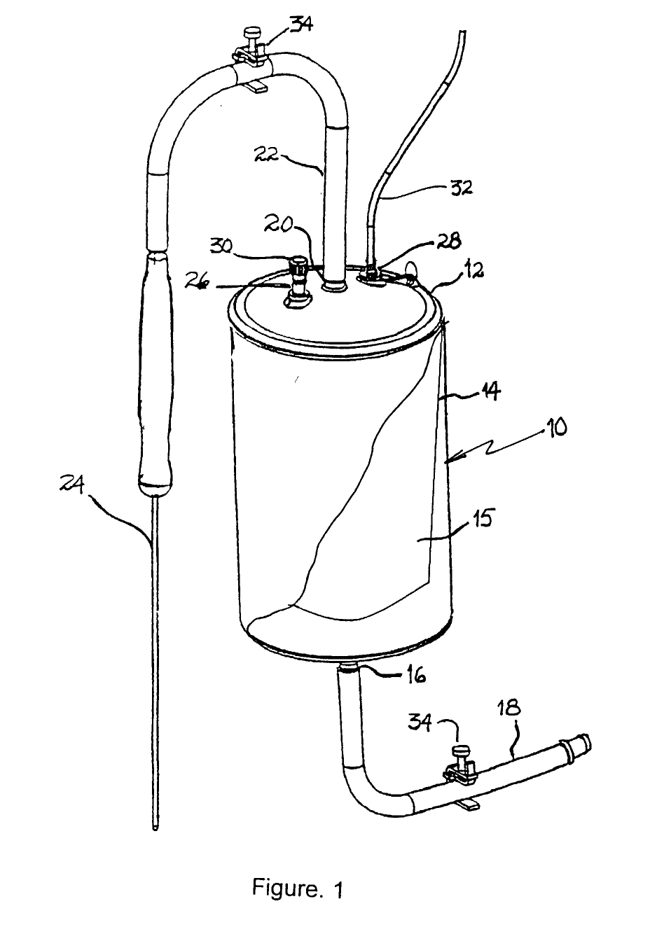

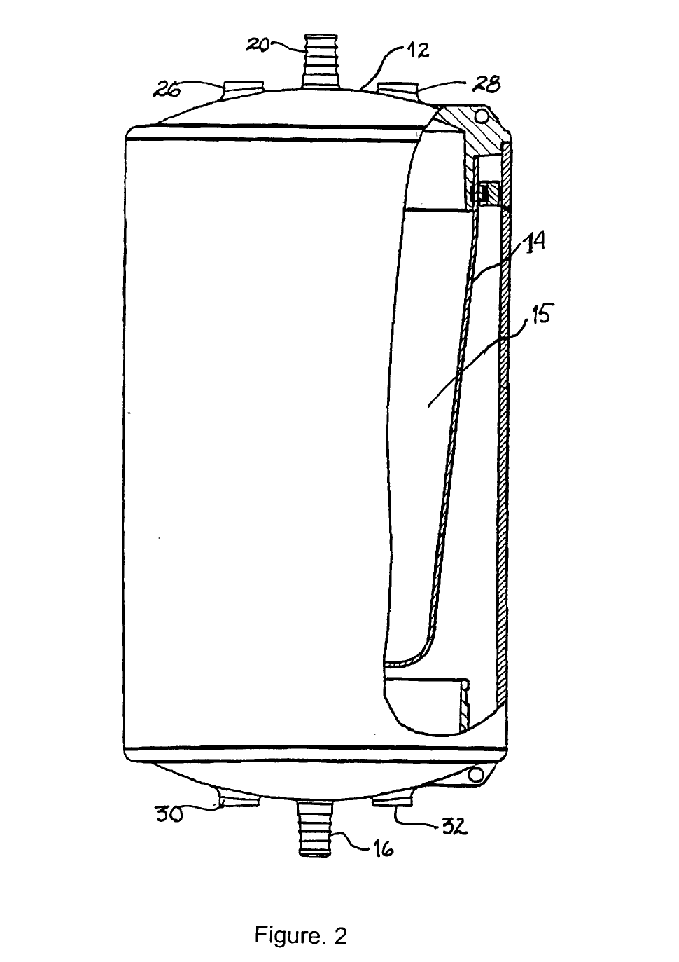

The following data were obtained from samples collected from 14 individuals. The collection was performed using suction-assisted lipoplasty with the tissue collected into the collection device described above and shown in FIGS. 1, 2. The device was then sealed, placed in an insulated shipping container along with two frozen gel packs and shipped to the processing facility. The tissue was then processed by washing with isotonic saline, and digesting using Liberase Blendzyme 1 collagenase (20 μg/ml; Roche Diagnostics, Indianapolis, Ind.). Released cells were concentrated by centrifugation at 400×g for 10 minutes at 4° C. and resuspended in 50 ml Plasma-Lyte A (Baxter Inc) supplemented with 5% Human Serum Albumin (American Red Cross Blood Service, Washington, D.C.) and 10% DMSO (Cryoserv; Edwards Life Sciences, Irvine, Calif.) in preparation for cryopreservation.

Cells were enumerated using a hemocytometer using Trypan Blue dye exclusion for visualization of viable cells. Stem cell content was determined using a CFU-AP assay in which 1,000 cells were plated in culture medium for 10 days at which time the plates were stained for cells expressing the alkaline phosphatase enzyme as a marker for osteogenic precursors. Information regarding the 14 samples are listed bellows:

| | Amount of | Post- | Total Viable | Total |
|---|---|---|---|---|
| | Tissue | Processing | Nucleated Cell | Content of |
| Sample | Collected | Cell | Content | CFU-AP |
| Number | (g) | Viability (%) | (x10⁸) | (x10⁶) |
| 1 | 1100 | 95 | 1.33 | 2.1679 |
| 2 | 800 | 62.2 | 1.18 | 2.36 |
| 3 | 400 | 81 | 2 | 0.4 |
| 4 | 650 | 78.1 | 1.6 | 10.4 |
| 5 | 1300 | 84.9 | 1.35 | 10.8 |
| 6 | 150 | 98 | 0.26 | 0.52 |
| 7 | 950 | 89 | 1.3 | 4.849 |
| 8 | 1200 | 71 | 1.1 | 1.958 |
| 9 | 700 | 79 | 1.02 | 0.4386 |
| 10 | 100 | 91.8 | 0.37 | 0.481 |
| 11 | 750 | 93.3 | 1.21 | 0.605 |
| 12 | 160 | 51 | 0.2 | 0.24 |
| 13 | 300 | 97 | 0.53 | 1.06 |
| 14 | 100 | 76.5 | 0.16 | 0.2656 |
| Average | 619 | 82 | 0.97 | 2,610,000 |
| Minimum | 100 | 51 | 0.16 | 240,000 |
| Maximum | 1,300 | 98 | 2.0 | 10,800,000 |

Therapeutic Utility

The above data demonstrates that the average CFU-AP (stem cell) dose obtained from liposuction was 2.6 million cells. Even the smallest collections (Samples 6, 10, and 14) yielded more than 176,000 CFU-AP.

Aliquots of the collected sample were cryopreserved as discussed above using 10% DMSO were then cooled from 4° C. to −50° C. at a controlled rate of −1° C. per minute. Once the samples reached −50° C., the cooling rate was increased to −10° C. per minute until −90° C. was reached. Thereafter, the aliquot samples were placed in the vapor space above liquid nitrogen in a sealed cryochamber.

The samples were maintained at −196° C. for an average of about 30 days.

Aliquots of cryopreserved cell populations were then thawed and assayed for recovery of nucleated cells and for retention of multi-lineage differentiation capacity.

Cells were thawed by placement of the cell container in a 37° C. waterbath an gentle agitation until such time as the consistency of the cells and medium within the container became slushy. The container was then immediately removed from the waterbath and placed on gel packs equilibrated to 4° C. The container was opened and the cells released into a wash solution containing tissue culture medium supplemented with 20% serum. Cells were then washed by centrifugation at 400×g at 4° C., the cell-free supernatant was discarded and the cells resuspended in tissue culture medium supplemented with 10% serum. Cells were counted and viability determined using Trypan Blue dye exclusion and counting using a hemocytometer. Cells were then placed in culture for determination of their ability to proliferate, to differentiate, and to evaluate the post-thaw content of stem cells (CFU-AP assay).

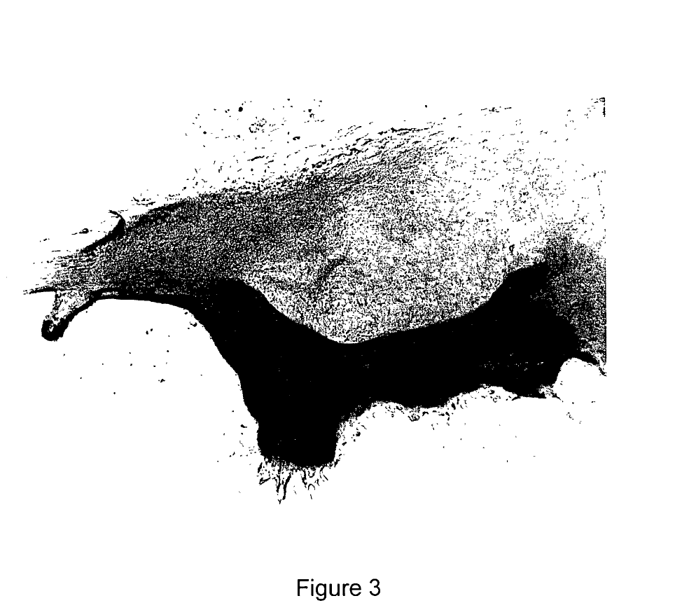

It was determined that 80.9% of cryopreserved cells were recovered after thawing. These cells showed no loss of ability to differentiate along the adipocytic lineage (103% of pre-freeze), osteoblastic differentiation (105% of pre-freeze), and neuronal differentiation (97% of pre-freeze). Quantitative determination of chondrogenic differentiation was not possible due to the nodular structure of the cultures. However, on a qualitative level no loss of chondrogenic potential was observed following cryopreservation. An example of a sample of cryopreserved and thawed cells is shown in FIG. 3.

### EXAMPLE 2

The following data is cited for the purpose of demonstrating utility of the process as applied to a particular sample of adipose tissue.

| Sample ID#: | SVF 94 |
|---|---|
| Donor Gender: | Female |
| Donor Age: | 68 |

Adipose Tissue Collection

Amount of Tissue Collected: 400 ml

Collection Method: Suction-Assisted Lipoplasty

Tissue Receptacle Type (Collection Device): Sterile Collection Device described above and shown in FIGS. 1 and 2.

Duration of Collection: approx 2 hours

Data Collected at Time of Collection:

Date and Time of Lipoplasty Procedure

Physician Identifier

Donor Identifier (linked to Bar Code Identifier)

Current Medications

Menopausal Status

Smoker or Non-smoker

Previous Surgeries

Previous Lipoplasty

General Health History

Shipment of Tissue to the Processing Site

Packing for Shipment: Collection device sealed in double plastic bag

Duration of Shipment: 2.5 hours

Temperature of Sample During Shipment (measured at receipt): 4° C.

Tissue Processing

Type of System Employed: Closed, sterile fluid pathway

Tissue Washing Solution: isotonic saline (temp. 38° C.)

Volume of Washing Solution Employed: 1,500 ml

Wash Fluid Collection Mechanism: 3 L plasma transfer bag (attached by sterile docking)

Method of Tissue Digestion: Enzymatic/Collagenase: Blendzyme 1, Roche Diagnostics; Used at 20 μg/ml

Tissue Digestion Time: 60 mins

Tissue Digestion Volume: 600 ml

Released Cell Collection Mechanism: 600 ml plasma transfer bag

Cell Concentration Mechanism: Centrifugation in Sorvall RC3 Centrifuge at 400×g, for 10 minutes at 4° C.

Waste Fluid Collection Mechanism: Expression of supernatant fluid into 600 ml Plasma Transfer Bag (Charter Medical)

Cell Suspension Medium: Plasma Lyte A®, (Baxter Inc) supplemented with 5% Human Serum Albumin; total volume 52 ml

Cryoprotectant: Cryoserv DMSO (Edwards Life Science) 5.75 ml added

Cryopreservation Procedure

Mechanism for Adding Cryoprotectant: Added using syringe pump at 0.25 ml/min. Cells pre-cooled to 4° C.; temperature maintained during addition of cryoprotectant using Insul-Ice (Polyfoam Packers) pre-conditioned to 4° C.

Cryopreservation Container: Stem Cell Freezing Bags (Pall Medical) (two bags used) placed within Aluminum outer containers (Pacific Sciences)

Cryopreservation Mechanism: MVE KryoSave using computer-controlled cooling of the cryopreservation chamber according to the following program:

| | |
|---|---|
| Start Temperature: | 4° C. |
| Cooling Rate A: | −1° C./min |
| Cooling Range A: | 4° C. to −50° C. |
| Cooling Rate B: | −10° C./min |
| Cooling Range B: | −50° C. to −90° C. |
| End (hold) Temperature: | −90° C. |

Cryostorage Conditions: Vapor phase of MVE 1841 Liquid Nitrogen storage tank

Cell Testing

Cell Counting Mechanism: Hemocytometer and manual count

Final Yield of Cells: 4.5×10⁷ viable nucleated cells

Cell Viability: Trypan Blue dye exclusion. 80% Viable Cells

CFU-AP Content: 7.7 CFU-AP/1,000 nucleated cells 345,375 total CFU-AP

Sterility Assay: 10 ml of supernatant solution added to Aerobic tube and 10 ml to Anerobic tube of BacT_Alert sterility testing system. Cultures maintained for 14 days.

Sterility results: No growth of microorganisms detected

Post-Thaw Analysis of Cryopreserved Cells

Stem Cell Assay (CFU-AP)

Pre-Freeze: 153.5 CFU-AP per 20,000 cells plated

Post-Thaw: 146 CFU-AP per 20,000 cells plated

Post-thaw recovery of CFU-AP: 95%

Based on the initial yield of CFU-AP=345,375, the post-thaw yield (95% recovery)=328,106

Cell Proliferation

20,000 cells were plated in stem cell tissue culture medium (DMEM supplemented with 10% fetal calf serum and antibiotics) and incubated in a humidified atmosphere of air +5%CO2 at 37C. for 14 days. Cells were then removed by brief trypsinization and cell number/yield determined using a hemocytometer.

| | | |
|---|---|---|
| Pre-Freeze: | Cell output | 2.2 ± 0.1 × 10⁴ nucleated cells |
| Post-Thaw: | Cell Output | 2.4 ± 0.4 × 10⁴ nucleated cells |

Summary: No statistically significant difference in proliferative capacity.

Test showed that the cryopreserved and thawed cells had substantially the same viability as the same batch of cells tested prior to preservation.

The cells are shown to retain full function after thawing with no loss of ability to differentiate into chondrocytes (cartilage), osteoblasts (bone), neuronal cells (nerve), and adipocytes (fat) in cell culture.

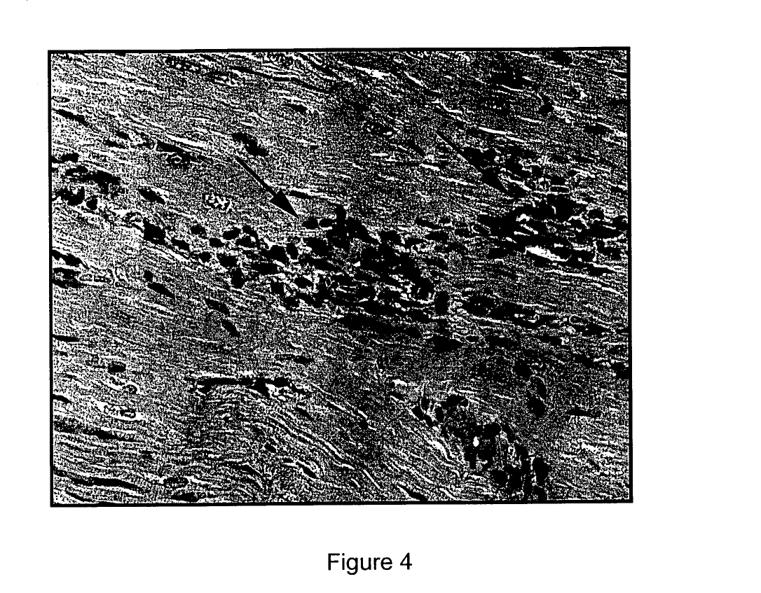

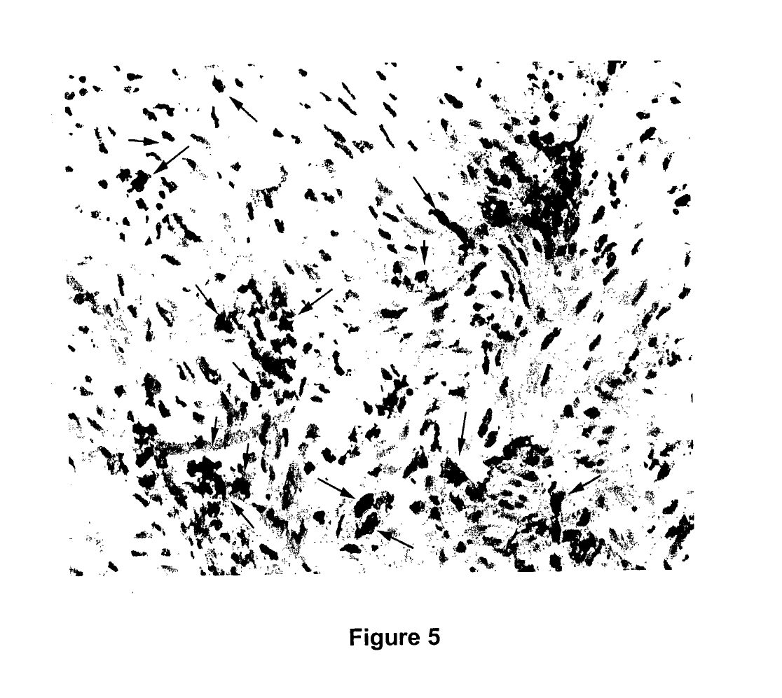

In vivo differentiation of these cells is also demonstrated in the rat model using cryopreserved rat adipose tissue-derived cells implanted into the heart of another rat (FIG. 4). Harvesting of the thawed cells one week post-implantation showed that they retained the ability to proliferate and differentiate in the receipient heart. Similar results were obtained following implantation of thawed human adipose tissue-derived cells into the bladder of an immunodeficient mouse with evidence of differentiation of the human cells (FIG. 5) into smooth muscle-like cells. This is in contrast to the failure of previous attempts to successfully cryopreserve adipose tissue-derived cells using cryostorage of intact adipose tissue (Lidagoster, M I, Cinelli P B, Levee E M and Sian C S “Comparison of Autologous Fat Transfer in Fresh, Refrigerated, and Frozen Specimens: an Animal Model. Ann Plast Surg. 44, P512-515 (2000)).

Recovery of Matrix Material

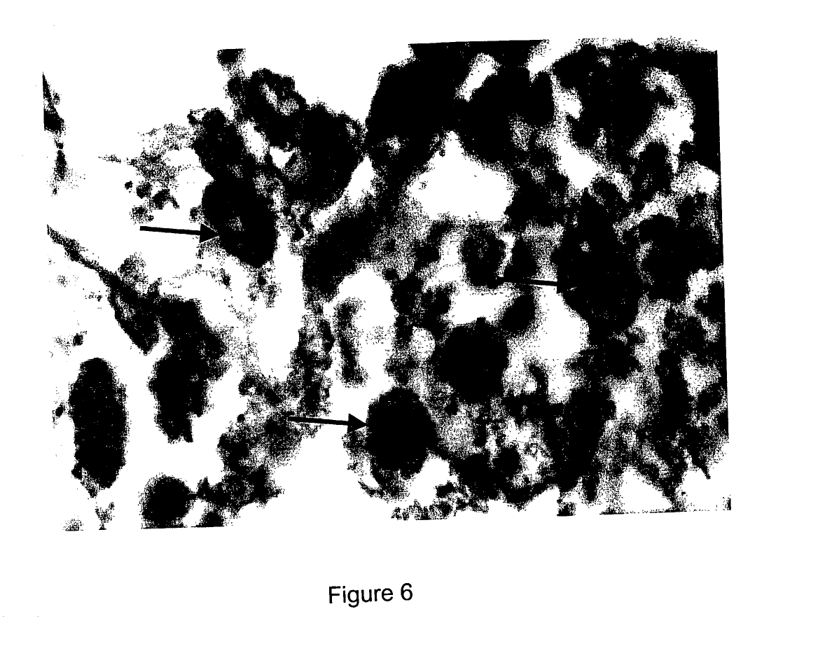

Recovery of matrix material will depend on the specific means used to store the material. These may include options as varied as adding a liquid vehicle to material stored in a syringe to thawing a material that is an intermediate in manufacture followed by continuation of the processing. Utility of stored matrix is demonstrated in FIG. 6 in which stored matrix material was recovered and used as a scaffold for in vitro generation of adipose tissue. FIG. 6 shows generation of mature, lipid-laden adipocytes (arrows) on the matrix scaffold.

Post-Storage Inspection of Cells and Matrix Material

Cells and material intended for clinical use can be inspected and/or tested prior to clinical application. In performing such activities care should be taken to preserve the viability of the thawed cells.

Potential tests include, but are not limited to, measurement of cell number and viability using trypan blue dye exclusion or similar technology, and confirmation of the identity of the cell donor by DNA fingerprinting or similar approach. In a preferred embodiment, a portion of the cryopreserved material sealed within the sterile, tubing leading to the cryostorage bag may be excised from the bag without interfering with, thawing, or exposing the contents of the bag to the environment. The cells sealed in this length of tubing may be thawed and inspected in advance of thawing the full contents of the bag. By this means, testing can be performed and the identity of the donor and cells confirmed before the main body of cells is thawed.

In addition, any or all of the inspections and tests listed above for cells at the end of processing (for example immunophenotyping or clonogenic culture) may be employed.

Post-Storage Manipulation of Cells and Material Prior to Clinical Application

The nature of the stem and progenitor cell populations within adipose tissue is such that they can be used for numerous applications including tissue engineering and gene medicine. Certain of the particular applications within these areas will require the manipulation of these cell populations prior to their placement within a recipient or use in research.

Examples include, but are not limited to, cell culture to increase the number and/or purity of the cell population, enrichment or purification of subpopulations, introduction of genetic material into the cells, or cell culture-based differentiation of the cell population to promote acquisition of a desired phenotype or function.

Application of a Quality System to Cell and Matrix Banking

An integrated quality system is an optional part of this invention. Such a system would cover all aspects of the invention to ensure control of the process and the minimization of the opportunity for error.

Although deemed optional the practice of this invention in the absence of a quality system would eventually lead to reduced quality, sample identification errors, and other problems.

Professional bodies such as the American Association of Tissue Banks and American Association of Blood Banks have requirements for the specifications of quality systems applicable to their areas of interest and which have considerable value in the context of this invention. Similarly, the ISO 9000 series of standards established by the International Organization for Standards, although necessarily generic, can be applied to this invention. Sample governmental regulations are included in regulations covering Good Tissue Practices released by the US Food and Drug Administration.

Some components of a quality system applicable to the practice of this invention include, but are not limited to, the following:

process control with procedures for each aspect of tissue handling (from first donor contact with the program through tissue collection, receipt at the processing facility, processing, testing, storage, and release)

cell population/matrix material product labeling throughout manufacture

organization, administration and oversight of the program including designation of persons responsible for development and implementation of the quality system

selection, training, and education of staff

selection, purchase, inspection, and use of supplies

selection, purchase, inspection, maintenance, and use of equipment

supplier evaluation

design and manufacture of products manufactured exclusively by or for the program

document and record creation, modification, and control

facilities

process improvement

error/incident detection

documentation and response to customer complaint

development of product and process specifications

safety

development and implementation of an internal program to evaluate compliance with the quality system

Practice of this invention may include none, some, or all of the above optional components.

The adipose tissue-derived stem and progenitor cell populations and the matrix material of the present invention have a multiplicity of potential therapeutic, structural, and cosmetic applications. Cells and/or matrix material for such applications may be provided from the individual in whom they are to be used (autologous applications), or from a related or unrelated donor (allogeneic applications). In a preferred embodiment of this invention the cell population the cells and/or matrix material are applied in autologous applications in which they are returned to the person from whom the adipose tissue was obtained. This approach avoids the use of anti-rejection medications and reduces the risk of rejection of the tissues and of introduction of an infectious agent. This approach is viable in conditions where the stem cell and progenitor cell populations and the matrix material are not a part of the underlying condition for which the cells are intended. For example, autologous stem and progenitor cells may be suitable for promoting repair of myocardial tissue or for cartilage repair. However, in the absence of gene transfer, autologous stem and progenitor cells derived from adipose tissue would NOT be appropriate for use in bone repair for a patient with Osteogeneis Imperfecta a genetic disorder of bone production. In such a setting allogeneic stem and progenitor cells from a related or unrelated donor may be preferred.

Alterations and variations of this invention may be made without departing from the spirit and scope thereof. The specific examples and embodiments contained in this disclosure are provided by way of example only and the invention is limited only by the terms of the appended claims.

For example, while the preferred collection procedure utilizes a collection device with a permeable membrane therein for initial separation of desired components from discardable component, the liposuction aspirate from a single patient may all be collected in a single collection container and all the collected material then transported to a location for separation and isolation of the stem cells, progenitor cells and matrix material and any other desired components prior to cryopreservation.

Still further, the invention also contemplates collecting, transporting and cryopreserving the total liposuction aspirate. The stem cells, progenitor cells, matrix material, and any other desired components may then be recovered and separated, using the procedure described above, at a later time following reheating of the aspirate to a suitable processing temperature, such as at or above 4° C. but preferably below body temperature.

We claim:

1. A process for recovering and preserving stem cells, progenitor cells and matrix material from adipose tissue for therapeutic applications comprising:
a. vacuum aspirating adipose tissue into a cell collection portion of a vessel, the cell collection portion being separated from the vessel surrounding the cell separating portion by a permeable membrane, the permeable membrane retaining desired components of the adipose tissue within the cell collection portion and the allowing discardable components to pass therethrough for disposal;
b. separation of the material retained in the collection portion and washing of said separated material to remove any discardable components and separating matrix material and an adipocyte-depleted cell population therefrom;
c. cryopreserving and cryostoring one or more of the the desired portions of the adipocyte-depleted cell population and the cell matrix.

2. The process of claim 1 wherein the desired components comprise a population of cells that includes stem cells, progenitor cells and matrix material and the discardable components comprise tumescent solution, saline, blood, blood clots, free lipids, and mature adipocytes.

3. The process of claim 1 wherein the permeable membrane is a filter material of defined porosity.

4. The process of claim 1 wherein the separation and washing procedure comprises isolating the material retained in the collection portion, disaggregation of the said isolated material and placing the disaggregated material in an isotonic buffered solution, stem cells and progenator cells thereby separating from free lipid and adipocytes, and removing the free lipid and adipocytes.

5. The process of claim 4 wherein the disaggregation is accomplished by exposing the material in the collection portion to proteolytic enzymes, mechanical disaggregation or a combination thereof.

6. The process of claim 5 wherein the disaggregation is accomplished by exposing the material in the collection portion to collagenase, trypsin, papain, DNase or lipase or mincing or shearing action or a combination thereof.

7. The process of claim 5 wherein the stem cells and progenitor cells are separated from the discardable components by centrifugation or buoyant density flotation.

8. The process of claim 1 wherein the adipocyte-depleted cell population is concentrated from other materials by centrifugation, spinning membrane separation, differential adherence, or elution to solid phase structures, and fluorescence activated cell sorting or a combination thereof.

9. The process of claim 8 wherein solid phase structures are anti-body covered beads.

10. The process of claim 8 further including separating the adipocyte-depleted cell population into a stem cell rich phase and a progenitor cell rich phase.

11. Mammalian stem cells suitable for delivery to mammals of the same species for therapeutic purposes comprising stem cells separated from adipose tissue, said stem cells having been cryopreserved and thawed prior to use.

12. The process of claim 1 wherein cryopreserving comprises mixing the adipocyte-depleted cell population with one or more cryoprotective additives, cooling the mixture so produced at a constant cooling rate of from about −1° C. to about −3° C. until the mixture reaches −50° C. and placing the cooled mixture in a storage vessel containing liquid nitrogen.

13. The process of claim 12 wherein the cryoprotective additives are selected from the group consisting of permeating, non-permeating cryoprotectants and combinations thereof.

14. The process of claim 13 wherein the permeating cryoprotectants is dimethyl sulfoxide, glycerol, 1,2-propanediol or combinations thereof and the non-permeating cryoprotectant is autologous plasma, human serum, hydroxyethyl starch, albumin, polyvinyl pyrrolidone and combinations thereof.

15. The process of claim 1 wherein
a. the adipocyte-depleted cell population is prepared as a cell suspension in a 5% human serum albumin solution,
b. the suspension is cooled to 4° C.,
c. dimethyl sulfoxide is slowly added to the cooled suspension while maintaining the suspension at 4° C. to produce a final DMSO concentration of from about 5 to about 20% by volume DMSO,
d. the cell suspension is cooled at a rate of about −1° C. per minute until a temperature of −50° C. is obtained,
e. the cell suspension is cooled at a rate of about −10° C. per minute until a temperature of −90° C. is obtained, and
f. the cell suspension is placed in the vapor phase of a liquid nitrogen storage vessel.

16. The process of claim 1 wherein the adipocyte-depleted cell population is separated into more than one portion before cryopreservation.

17. The process of claim 15 wherein the final DMSO concentration is 10% by volume.

18. The process of claim 1 further including the step of recovering the cryopreserved adipocyte-depleted cell population comprising slowly elevating the temperature thereof to about 4° C.

19. The process of claim 1 wherein the matrix material is separated from any other materials recovered, is freeze dried to a residual water content of from about 0.5% to about 5.0% water by volume and is stored at or below about 25° C.

20. A therapeutic composition comprising stem cells in a carrier solution for delivery to a patient, said stem cells recovered from adipose tissue, separated from other cellular material, cryopreserved and then thawed, said stem cells viable for therapeutic or cosmetic applications.

21. The therapeutic composition of claim 20 further including progenitor cells recovered from adipose tissue and subjected to the same processing as the stem cells.

22. A cryopreserved therapeutic composition comprising viable stem cells derived from adipose tissue of a single donor in an amount of a cryopreservative sufficient to preserve an adequate level of viability of said stem cells, said stem cells being present in amounts sufficient to differentiate into various cell types and to proliferate after said stem cells are thawed and the cryopreservative is removed.

23. The cryopreserved therapeutic composition of claim 22 further including viable progenitor cells.

24. A process for treating a mammalian subject diagnosed as having a disease state treatable by delivery of stem cells and/or progenitor cells comprising delivering to the body of that individual previously cryopreserved stem cell, progenitor cells or a combination thereof, wherein the cryopreserved stem cells and progenitor cells have been previously harvested from the adipose tissue of that individual.

25. The process of claim 24 wherein the cryopreserved stem cell and progenitor cells are recovering and preserving from adipose tissue of the mammalian subject by the process of:
a. vacuum aspirating adipose tissue into a cell collection portion within a vessel, the cell collection portion being separated from the vessel surrounding the cell separating portion by a permeable membrane, the permeable membrane retaining desired components of the adipose tissue within the cell collection portion and the allowing discardable components to pass there through for disposal;
b. separation of the material retained in the collection portion and washing of said separated material to remove any discardable components and separating an adipocyte-depleted cell population therefrom; and the cryopreserving and cryostoring adipocyte-depleted cell population, being removed from storage, heated to at least about 4° C. and substantially all of any cryopreservative present therein is removed prior to delivery of the cryopreserved adipocyte-depleted cell population to the individual.
